(19) United States
(12) Patent Application Publication
(10) Pub. No.: US 2009/0197963 A1
(43) Pub. Date: Aug. 6, 2009

(54) Method and compositions for suppressing appetite or treating obesity

(76) Inventor: Dan Llewellyn, Carlstadt, NJ (US)

Correspondence Address: KLAUBER & JACKSON, 411 HACKENSACK AVENUE, HACKENSACK, NJ, 07601, US

(21) Appl. No.: 12/322,108

(22) Filed: Jan. 29, 2009

Related U.S. Application Data
(60) Provisional application No. 61/063,378, filed on Feb. 1, 2008.

(51) Int. Cl. A61K 31/275

(52) U.S. Cl. 514/579

(57) ABSTRACT

The present invention provides a method for reducing the appetite in a mammal by contacting the mammal with an amount of a noxious smelling agent effective in reducing appetite or by contacting the mammal with an agent effective in inhibiting the effect of another agent in stimulating appetite. Also, the present invention provides a method for treating obesity in a mammal by contacting the mammal with an amount of a noxious smelling agent effective in reducing appetite or with an agent effective in inhibiting the effect of another agent in stimulating appetite. Similarly, the present invention provides compositions and kits for both reducing appetite and treating obesity. In some instances, an isonitrile or an isocyanide is present in the composition or kit.

### CROSS REFERENCE TO RELATED APPLICATIONS

Priority is claimed under 35 USC §119(e) to Provisional Patent Application No. 61/063,378, filed Feb. 1, 2008.

### FIELD OF THE INVENTION

This invention relates to methods for suppressing appetite and therefore advancing weight loss. The methods are particularly useful for treating obesity.

### BACKGROUND OF THE INVENTION

Obesity is a disorder characterized by the accumulation of excess fat in the body. Obesity is emerging as a global problem and is a major factor for a number of co-morbidities such as coronary heart disease, hypertension, non-insulin dependent diabetes mellitus, pulmonary dysfunction, osteoarthritis and certain types of cancer.

Obesity has been defined in terms of body mass index (BMI). BMI is calculated as weight (kg)/[height(m)]². In addition to those individuals who satisfy a strict definition of medical obesity, a significant portion of the adult population is overweight. These individuals would also benefit from the availability of an effective weight-loss composition. Current products to suppress appetite and control weight are generally drugs with undesirable side effects. The main factor causing the development of obesity is a positive energy balance through the decreased activity and increased energy intake. Weight loss and loss of body fat can thus be achieved by reducing food intake and/ or increasing energy expenditure.

Studies show that weight tends to decline after a certain age. The reason for the decline in weight with aging has been attributed to the normal decline in the taste and smell senses. The smell of food alone has been demonstrated to increase pancreatic polypeptide within the first 3 minutes and to increase colonic pressure. The sight and smell of food increase insulin secretion in the first 20 minutes and this rise in insulin is blocked by atropine, suggesting that the rise is vagally mediated. Patients with anorexia have been shown to have a diminished sense of smell and in case reports, a diminished sense of taste and smell have been associated with weight loss. The smell of food also increases appetite and food intake in restrained eaters.

### SUMMARY OF THE INVENTION

In a first aspect, the present invention provides a method for reducing the appetite in a mammal comprising contacting a mammal with an amount of a noxious smelling agent effective in reducing appetite or by contacting a mammal with an amount of an agent effective in inhibiting the effect of another agent in stimulating appetite. In a preferred embodiment, the noxious smelling agent is an isonitrile or an isocyanide. The noxious smelling agent may be a phenyl isonitrile or an alkyl isonitrile. In preferred embodiments, the mammal is contacted with the agent by nasal stimulation. That is, the agent is brought in proximity to the nose such that the sense of smell is stimulated. In some embodiments the mammal is contacted with more than one noxious smelling agent either sequentially or concurrently or with one or agent effective in inhibiting the effect of another agent in stimulating appetite, either sequentially or concurrently.

In a preferred embodiment, the amount of noxious smelling agent or agent effective in inhibiting the effect of another agent in stimulating appetite is between about 1 mg and about 1000 mg, preferably about 2 mg and 500 mg, or between 3 mg and 300 mg or between 4 mg and 200 mg. In another embodiment, the method includes instilling a noxious smelling agent or an agent effective in inhibiting the effect of another agent in stimulating appetite into the nasal passageways to effect contact with olfactory nerves. In some embodiments, the instilling step can be repeated at least two, three, four, five, six or more times within about one day, one week or even one month.

The contacting may be performed for a period of at least about 0.5 seconds, 1 second, 2 seconds, 3 seconds, 4 seconds or even 10 or more seconds. In some embodiments where the agent is placed proximal to the nose or implanted in the nasal passages, the contacting may be performed for a period of at least about an hour, 2 hours, 12 hours, 24 hours, 2 days, 5 days, a week, 10 days, 2 weeks or even a month. The agent may be provided, for instance, in a liquid or fast melt form in any suitable container, or it may be provided in a gaseous form in any suitable container. A suitable container may be, for instance, a jar or vial having a lid or top that may be temporarily or permanently removed. For instance, a screw cap jar or container is in many instances suitable as a container so that the duration or the contact may be controlled as well as the intensity. That is, a suitable container may normally be adjusted in proximity to the nose so that the intensity of stimulation of the smelling sense may be controlled.

In a second aspect, the invention provides a composition for reducing appetite in a mammal, comprising a noxious smelling agent or an agent effective in inhibiting the effect of another agent in stimulating appetite. The agent may be provided in a liquid form, in a fast-melt delivery form, or may be provided absorbed onto a solid such as a pad, cloth, or foam. The noxious smelling agent may be a phenyl isonitrile or an alkyl isonitrile. In preferred embodiments, the mammal is contacted with the agent by nasal stimulation. That is, the agent is brought in proximity to the nose such that the sense of smell is stimulated. In some embodiments the mammal is contacted with more than one noxious smelling agent or with more than one agent effective in inhibiting the effect of another agent in stimulating appetite either sequentially or concurrently, or by both one or more noxious smelling agent and with one or more than one agent effective in inhibiting the effect of another agent in stimulating appetite either sequentially or concurrently. In some embodiments, the concentration of the agent is between about 1 ppm and about 1000 ppm, between 2 ppm and 500 ppm, between 3 ppm and 400 ppm, between 4 ppm and 300 ppm or between 5 ppm and 200 ppm.

In a third aspect, the invention provides a method of treating obesity in a mammal including the step of contacting the mammal with a noxious smelling agent sufficient to stimulate the sense of smell in the mammal or contacting the mammal with an agent effective in inhibiting the effect of another agent in stimulating appetite. The noxious smelling agent may be an isonitrile or an isocyanide. The noxious smelling agent may be, for instance, a phenyl isonitrile or an alkyl isonitrile. In preferred embodiments, the mammal is contacted with the noxious smelling agent or the agent effective in inhibiting the effect of another agent in stimulating appetite by nasal stimulation. That is, the agent is brought in proximity to the nose such that the sense of smell is stimulated. In some embodiments the mammal is contacted with more than one noxious smelling agent or with one or more agent effective in inhibiting the effect of another agent in stimulating appetite either sequentially or concurrently. Likewise, in some embodiments, the mammal is contacted with one or more than one noxious smelling agent and with one or more than one agent effective in inhibiting the effect of another agent in stimulating appetite either sequentially or concurrently. The contacting may be performed for a period of at least about 0.5 seconds, 1 second, 2 seconds, 3 seconds, 4 seconds or even 10 or more seconds. In some embodiments where the agent is placed proximal to the nose or implanted in the nasal passages, the contacting may be performed for a period of at least about an hour, 2 hours, 12 hours, 24 hours, 2 days, 5 days, a week, 10 days, 2 weeks or even a month. The agent may be provided, for instance, in a liquid or fast melt form in any suitable container, or it may be provided in a gaseous form in any suitable container. A suitable container may be, for instance, a jar or vial having a lid or top that may be temporarily or permanently removed. For instance, a screw cap jar or container is in many instances suitable as a container so that the duration or the contact may be controlled as well as the intensity. That is, a suitable container may normally be adjusted in proximity to the nose so that the intensity of stimulation of the smelling sense may be controlled.

Further, in a fourth aspect, the present invention provides a kit for suppressing the appetite of a mammal or for treating obesity in a mammal comprising a noxious smelling agent sufficient to stimulate the sense of smell in the mammal or an agent effective in inhibiting the effect of another agent in stimulating appetite in the mammal. The agent may be provided in, for instance, liquid, fast melt or gaseous form, and the agent may be provided in a suitable container such as, for instance, ajar or vial having a lid or top that may be temporarily or permanently removed. The kit may optionally contain directions for using the agent alone or in conjunction with other agents or regimens for suppressing appetite or treating obesity. The directions may specify, for instance, means for contacting the agent with a mammal, frequency of contacting and duration of contacting.

### DETAILED DESCRIPTION OF THE INVENTION

By “noxious smelling agent” is meant any single substance such as an organic molecule, whether naturally occurring or synthetic, or combination of substances that when placed in reasonable proximity to the nose, stimulates the sense of smell to such a degree that a substantial number of subjects report the perceived smell as “unpleasant,” “foul” or “noxious,” or a substantial number of subjects report that the perceived smell is something to be avoided or experienced in only relatively short time periods. Specific substances included within the meaning of the term are isonitriles or isocyanides, for instance, phenyl isonitriles and alkyl isonitriles.

By “an agent effective in inhibiting the effect of another agent in stimulating appetite” is meant any single substance such as an organic molecule, whether naturally occurring or synthetic, or combination of substances that when placed in reasonable proximity to the nose, stimulates or inhibits the sense of smell to such a degree as to interfere with the ability of another agent stimulate appetite in a subject. Such an agent effective in inhibiting the effect of another agent in stimulating appetite may act for instance, to mask or cover the smell that might otherwise stimulate the appetite in a subject. Such an agent effective in inhibiting the effect of another agent in stimulating appetite need not necessarily provide a noxious or unpleasant odor. Such an agent may merely mask, cover up or neutralize an odor or smell that is normally active to stimulate the appetite in a subject. The odor or smell that is normally active to stimulate the appetite in a subject may be, for instance, the odor or smell of a food product.

The effect that the smell and taste of food has on the body and appetite is well documented. Therefore, manipulating or stimulating the sense of smell may directly impact the taste sensation and may directly impact the appetite. Manipulating or stimulating the sense of smell regularly, or upon perceiving hunger or prior to meals may be an effective strategy to decrease food intake. The present invention provides methods of suppressing the appetite and treating obesity by stimulating the olfactory neurosensory perception, e.g. the sense of smell with noxious smelling agent. With exposure to a noxious smelling agent and the resulting unpleasant smell sensed as a result, a reduced sensory perception of food may result or a direct sense of satiety or reduced sensation of hunger may result. Consequently, a subject may exhibit lower food intake. The amount of noxious smelling agent provided is sufficient to evoke only a short term response in many embodiments, e.g., an individual dosage of from about 1 ppm to about 1000 ppm of noxious smelling agent in an appropriate vehicle. In addition to drops or sprays for nasal administration, a fast melt composition may be used to release the odor only upon exposure to ambient air.

The present invention provides a method for behavior modification, i.e. overeating or eating more than is desired to maintain a certain weight, using olfactory stimuli. Different odors are provided either in liquid or gaseous form, either alone or with other substances, either in a container such as ajar or vial or on a patch or pad. The jar or vial may have a lid or cover that may be temporarily removed so as to allow intermittent contact with the noxious smelling agent for a specific period of time. The patch may have at least one odor, and in some instances has a dominant odor, i.e. an odor that is smelled most of the time. A subservient odor may also be provided which is only temporary in duration, that is, when released, its odor lasts for a short time, e.g. only long enough to provide a temporary smell sensation so as to modify appetite or the desire to eat, and then fades away to reveal the dominant odor once again. The subservient odor is in many instances a noxious smelling agent. The odors are in many instances organic solvent products of natural and synthetic aroma chemicals and the like which are well known in the art. The noxious smelling agent or agent effective in inhibiting the effect of another agent in stimulating appetite may be microencapsulated and placed on a patch. Whenever the urge to eat or to overeat is felt, the user may scratch the patch and release a subservient odor. This odor is then smelled and acts as an olfactory stimulus to suppress appetite. In some instances, where a patch is used, after a short period of time has passed, the subservient odor may fade, and the dominant odor may then be detectable. In such instances, a dominant odor that is preferably pleasant smelling may be provided while the subservient odor is a noxious smelling agent, preferably foul or fetid. In instances where the noxious smelling agent in provided in a container, jar or vial, whenever the urge to eat or to overeat is felt, the user may simply completely or partially remove the lid or cover of the container, jar or vial and release an odor of the noxious smelling agent.

The present invention provides an olfactory stimulus that assists in modifying behavior of a subject, such as suppressing appetite. The noxious smelling agent or agent effective in inhibiting the effect of another agent in stimulating appetite may be in the form of a microencapsulated chemical odorant that may be provided on a patch or pad, or the noxious smelling agent may be a liquid or a gas. One or more noxious smelling agents may be present in any composition that is provided. Likewise, one or more agents effective in inhibiting the effect of another agent in stimulating appetite may be present in any composition that is provided. The noxious smelling agent may be a natural or synthetic aroma chemical. Particular exemplary noxious smelling agents include isocyanides or isonitriles, such as, for instance, a phenyl isonitrile or an alkyl isonitrile. Isocyanides or isonitriles are organic compounds having the functional group R—N≡C. The CN functionality is connected to the organic fragment via the nitrogen atom, not via carbon as is found in the isomeric nitriles. The disagreeable odor of isocyanides or isonitriles is legendary. Exemplary noxious smelling agents may also include any of the following organic solvent products: Thione 220 SB-02-0333 IFF LB gas odorant; 2-phenylethyl mercaptan; Mangone 130456 IFF; Triethylamine; Phenyl acetic acid; IFF 2025-1503 i.e., “vomit smell”; N-Butyric acid; diethyldithiocarbanic acid and its sodium salt; Skatole 3-methylindole; Methyl disulfide (CH₃—S—S—CH₃) and Indol 2,3-Benzopyriole. The solvent for all of these may be, e.g. diethylphtholate, which has no odor.

This invention is illustrated in the Experimental Details section which follows. These sections are set forth to aid in an understanding of the invention but are not intended to, and should not be construed to limit in any way the invention as set forth in the claims which follow thereafter.

### EXAMPLE 1

Isocyanides may be synthesized by the reaction of primary amines with dichlorocarbene or by dehydration of a formamide with phosphorus oxychloride.

RNH₂+CCl₂+2 NaOH→RNC+2 NaCl+2 H₂O RNHC(O)H+POCl₃→RNC+“PO₂Cl”+2 HCl

The Hofmann isocyanide synthesis is a chemical test for primary amines based on their reaction with potassium hydroxide and chloroform as dichlorocarbene precursors to foul smelling isocyanides.

Isocyanides may also be synthesized by the reaction of organolithium compounds with oxazoles and benzoxazoles. The benzoxazole gets deprotonated at the 2-position by n-butyllithium. The lithium compound is in chemical equilibrium with the 2-isocyanophenolate which can be captured by an electrophile such as an acid chloride. Being an ester the formed isocyanate in the example above behaves uncharacteristically with reportedly a mild cherry smell. Also, isocyanides may be synthesized by the condensation of an amine with formic acid, yielding a formamide, and subsequently dehydrating this formamide.

We claim:

1. A method for reducing the appetite in a mammal comprising contacting the mammal with a noxious smelling agent effective to provide a smell sensation and thereby reduce appetite or contacting the mammal with an agent effective in inhibiting the effect of another agent in stimulating appetite.

2. The method of claim 1, wherein the noxious smelling agent is an isonitrile or an isocyanide.

3. The method of claim 1, wherein the amount of the agent is between about 1 mg and about 1000 mg.

4. The method of claim 1, further comprising instilling the agent into the nasal passageways to affect contact with olfactory nerves.

5. The method of claim 10, wherein the instilling step is repeated at least two times within about one month.

6. A composition for reducing appetite in a mammal, comprising a noxious smelling agent effective to provide a smell sensation and thereby reduce appetite or an agent effective in inhibiting the effect of another agent in stimulating appetite.

7. The composition of claim 6, wherein the concentration of the agent is between about 1 ppm and about 1000 ppm.

8. The composition of claim 6, wherein the agent is an isonitrile or an isocyanide.

9. A method of treating obesity in a mammal comprising contacting the mammal with an agent effective to provide a smell sensation and thereby reduce appetite.

10. The method of claim 9, wherein the agent is an isonitrile or an isocyanide.

11. A kit for suppressing the appetite of a mammal or for treating obesity in a mammal comprising a noxious smelling agent sufficient to stimulate the sense of smell in the mammal or an agent effective in inhibiting the effect of another agent in stimulating appetite.
